(19) United States
(12) Patent Application Publication
(10) Pub. No.: US 2017/0014432 A1
(43) Pub. Date: Jan. 19, 2017

(54) COMPOSITIONS AND METHODS FOR REDUCING CHRONIC LOW-LEVEL INFLAMMATION

(71) Applicant: Barry D. SEARS, Palm City, FL (US)

(72) Inventor: Barry D. SEARS, Palm City, FL (US)

(21) Appl. No.: 15/124,057

(22) PCT Filed: Mar. 6, 2015
(86) PCT No.: PCT/US15/19115

§ 371 Date: Sep. 7, 2016

Related U.S. Application Data
(60) Provisional application No. 61/952,297, filed on Mar. 13, 2014.
(60) Provisional application No. 62/067,221, filed on Oct. 22, 2014.

(51) Int. Cl. A61K 31/60; A61K 9/00; A61K 45/06; A61K 31/36; A61K 31/202

(52) U.S. Cl. CPC A61K 31/60; A61K 31/36; A61K 31/202; A61K 45/06; A61K 9/0014

(57) ABSTRACT

The invention relates to compositions and methods for reducing chronic low-level inflammation associated with chronic conditions. Specifically, the invention relates to compositions of hydrophobic derivatives of aspirin and/or hydrophobic derivatives of sesamol in combination with eicosapentaenoic acid (EPA) and docosahexaenoic acid (DHA), and their methods for reducing chronic low-level inflammation associated with chronic conditions including obesity, metabolic syndrome, diabetes, cardiovascular disease, cancer, auto-immune disorders, ocular and neurological disorders in a mammal.

### FIELD

The invention relates to compositions and methods for reducing chronic low-level inflammation associated with chronic conditions including obesity, metabolic syndrome, diabetes, cardiovascular disease, cancer, auto-immune disorders and neurological disorders in a mammal.

### BACKGROUND

Inflammation is like a double-edged sword. On one hand, it protects us from microbial invasion and allows our injuries to heal. On the other hand, if the initiation phase of the inflammatory response is too strong or is not shut down effectively, the result is chronic low-level chronic inflammation that begins to attack our organs leading to the acceleration of chronic diseases such as diabetes, heart disease, Alzheimer's disease and a vast array of other chronic conditions. It has been often believed in the past that inflammation simply dies out like a burning log, but we now know that the shutting down of the inflammatory response involves an active process known as resolution. Pro-resolution mediators, many of which are derived from omega-3 fatty acids, govern the regulation of the return of the inflammatory status to a homeostatic condition.

The first phase of the inflammatory response is the initiation phase mediated in part by pro-inflammatory hormones such as eicosanoids, which are derived from the omega-6 fatty acid arachidonic acid (AA). The second phase of the inflammatory response is the resolution phase mediated in part by pro-resolution mediators such as resolvins and other pro-resolution compounds, which are derived from omega-3 fatty acids such as eicosapentaenoic acid (EPA) and docosahexaenoic acid (DHA). Any imbalance between the initiation phase and resolution phase of the inflammatory response results in chronic low-level inflammation.

Chronic low-level inflammation has been associated with many chronic disease conditions including obesity, metabolic syndrome, diabetes, cardiovascular disease, cancer, auto-immune disorders and neurological disorders. Polyunsaturated omega fatty acids have been implicated to play a role in regulating these two significant, yet distinct phases of the inflammatory response.

Without adequate levels of omega-3 fatty acids in the target organs, the generation of many of these pro-resolution mediators is compromised. Although it is possible to synthesize these pro-resolution mediators, it is extremely costly and they must be injected. A preferable approach would be to maintain a reservoir of adequate levels of omega-3 fatty acid in every target tissue in the body as well as maintain a constant supply of these pro-resolution mediators in the blood to modulate on-going inflammatory responses. The invention described in this patent application provides such a solution to reduce chronic low-level inflammation by the enhancement of the pro-resolution phase of inflammation as well as reducing the intensity of the initiation phase of inflammation.

Methods described in this invention can reduce the production of AA formation as well as increase the formation of pro-resolution mediators such as resolvins derived from omega-3 fatty acids to accelerate the resolution of the acute inflammatory response by reducing chronic low-level inflammation thus significantly enhancing the therapeutic potential of products containing omega-3 fatty acids to treat a wide number of chronic disease conditions.

In situations of improperly regulated inflammation response results in chronic low-level inflammation. For example, the initiation phase can be too strong or the resolution phase may be too weak. Left untreated, this situation will result in chronic low-grade inflammation that can lead to a number of chronic conditions including obesity, metabolic syndrome, diabetes, cardiovascular disease, cancer, auto-immune disorders and neurological disorders. A more detailed listing of these conditions can be found in Table 1.

TABLE 1
Conditions That Can Be Improved By Reducing
Chronic Low-level Inflammation

Neurological disorders including:
Brain trauma (mild and severe)
Depression
Attention deficit hyperactivity disorder
(ADHD)/attention deficit disorder (ADD)
Parkinson's disease
Alzheimer's disease
Neuropathy
Ocular disorders including:
Age-related macular degeneration (AMD)
Dry eye syndrome
Optic nerve damage
Diabetic retinopathy
Auto-immune disorders including:
Type 1 diabetes
Rheumatoid arthritis
Multiple sclerosis
Lupus
Sorgen's disease
Sepsis
Cancer
Heart Disease
Osteoporosis
Metabolic Disorders including:
Type 2 diabetes
Metabolic syndrome
Non-alcoholic steatohepatitis (NASH)
Obesity
Asthma
Allergies
Gastrointestinal disorders including:
Colitis
Leaky gut syndrome
Skin disorders including:
Psoriasis
Inflammation caused by intense exercise

It has been shown that omega-3 fatty acids are weak inhibitors of the initiation phase of inflammation. In particular, EPA can inhibit the initiation phase by competing with AA for cyclo-oxygenase (COX) or lipo-oxygenase (LOX) enzymes that are necessary for the synthesis of pro-inflammatory eicosanoids. On the other hand, the real power of omega-3 fatty acids to treat chronic low-level inflammation is their critical role as substrates for the synthesis of the pro-resolution mediators that are necessary for the resolution of inflammation.

The potential of omega-3 fatty acids to modulate the inflammatory response can be significantly enhanced by either the acceleration of the resolution phase or the reduction of the levels of AA thereby reducing the initiation phase of inflammation, and ideally by a combination of both.

Since AA is produced by the enzyme Δ-5-desaturase (D5D), the synthesis and/or identification of specific inhibitors of D5D can help treat, moderate, and prevent inflammation. U.S. Pat. No. 6,172,106, the entire disclosure of which is incorporated by reference herein, has identified sesamol as a specific inhibitor of D5D. Further, the related U.S. patent application Ser. No. 13/893,803, filed on May 14, 2013, now issued U.S. Pat. No. 8,987,325, the entire content of which is herein incorporated by reference, has identified hydrophobic sesamol derivatives as specific inhibitors of D5D that are less toxic and compatible with therapeutic omega-3 fatty acids formulations as compared to sesamol itself.

A problem associated with an omega-3 fatty acid-mediated pro-resolution treatment regime is that the levels of omega-3 fatty acids must be very high. This could potentially lead to increased bleeding and depression of the initiation phase of inflammatory response. As a result, inconsistent therapeutic results are often observed using omega-3 fatty acids to treat the variety of chronic conditions shown in Table 1. Hence, there is a need to develop novel compositions and methods that overcome the obstacle for the therapeutic use of omega-3 fatty acids in the regulation and reduction of chronic low-level inflammation by controlling both the initiation and resolution phases of the inflammatory response.

### SUMMARY

The present invention relates to compositions of hydrophobic derivatives of aspirin and/or sesamol that can be precisely matched to the correct daily dosage of omega-3 fatty acids for the individual that allows for the maximum generation of pro-resolution mediators without the associated side effects such as increased bleeding when taken on a chronic basis to treat and manage low-level inflammation.

The clinical benefits of the present invention would allow precise administration of the compositions coupled with an easy-to-measure blood parameter to maintain adequate resolution of inflammation regardless of the cause. In addition, the dose of the administration can be individualized based on the type and severity of diseases, as well as gender and age of the subject, and can be used as either a therapeutic or prophylactic intervention.

The composition of the present invention consists of a defined amount of hydrophobic aspirin analog dissolved in given amount of a refined omega-3 concentrate containing high levels of both eicosapentaenoic acid (EPA) and docosahexaenoic acid (DHA). EPA and DHA are substrates required to generate many of the most powerful pro-resolution mediators (resolvins, maresins, and protectins) that are required for the resolution phase of the inflammatory response. The addition of a defined amount of hydrophobic aspirin to increase the levels of pro-resolution mediators will generate an increased production of pro-resolution mediators from these omega-3 fatty acids circulating in the blood. This overcomes the need for injection of synthetic resolvins on an acute basis to reach a target organ as each organ in the body and ensures the mammal will have a sufficient reservoir of omega-3 fatty acids in their membranes to maintain the constant generation of the necessary pro-resolution mediators.

The appropriate level of composition to be administered to a mammal is determined by the AA to EPA ratio in the blood. AA is the primary driver of the eicosanoids that are important in the initiation process of inflammation. EPA is a stereochemical competitor to AA for access to a variety of enzymes that produce pro-inflammatory eicosanoids necessary for the initiation and continuation of the inflammatory response as well as substrate to make pro-resolution mediators such as resolvins. Thus the AA/EPA ratio in the blood allows one to optimize the level of the composition to be administered for either the therapeutic treatment or prophylactic maintenance to prevent excess inflammation with a precision simply not available by giving oral aspirin and omega-3 fatty acids separately, or by injections of synthesized pro-resolution mediators. The effect of the hydrophobic analog of aspirin to increasing resolution phase of inflammation can be further enhanced by the addition of a hydrophobic analog of sesamol that reduces the production of AA thus reducing the levels of omega-3 fatty acids needed for resolution.

It has been unexpectedly discovered that low levels of hydrophobic aspirin or the combination of hydrophobic derivatives of sesamol and hydrophobic aspirin with eicosapentaenoic acid (EPA) and docosahexaenoic acid (DHA) provides a method to overcome the inconsistent therapeutic results using omega-3 fatty acids by achieving enhanced resolution with low levels by the addition of these hydrophobic compounds are combined with omega-3 fatty acids.

In one aspect, the invention provides a composition comprising hydrophobic derivatives of aspirin and/or hydrophobic derivatives of sesamol and aspirin in combination with eicosapentaenoic acid (EPA) and docosahexaenoic acid (DHA), for reducing chronic low-grade inflammation associated with chronic conditions including obesity, metabolic syndrome, diabetes, cardiovascular disease, cancer, auto-immune disorders and neurological disorders in a mammal.

The hydrophobic aspirin will enhance the production of group of pro-resolution mediators derived from omega-3 fatty acids (resolvins, protectins, maresins, etc.) that are critical in the resolution process of inflammation. The addition of hydrophobic sesamol to the hydrophobic aspirin in the composition can dampen the strength of the inflammatory response by reducing the production of arachidonic acid that is the building block of many pro-inflammatory eicosanoids.

The hydrophobic analogs of aspirin and/or sesamol allow for their compatibility with the hydrophobic omega-3 fatty acids to produce a highly defined formulation with the exact ratio of the hydrophobic analogs to omega-3 fatty acids that will maximize the resolution of chronic low-level inflammation without the possibility to administering too much of either the omega-3 fatty acids or aspirin to increase bleeding times in the mammal. Likewise the hydrophobic sesamol allows a precise reduction of the intensity of the initiation phase of the inflammatory response by decreasing production of arachidonic acid.

In another aspect, the invention provides a method for reducing chronic low-grade inflammation associated with chronic conditions including obesity, metabolic syndrome, diabetes, cardiovascular disease, cancer, auto-immune disorders and neurological disorders in a mammal, comprising administering to a mammal a composition comprising hydrophobic derivatives of aspirin and/or hydrophobic aspirin and hydrophobic sesamol in combination with therapeutic levels of eicosapentaenoic acid (EPA) and docosahexaenoic acid (DHA).

In one embodiment, the level of or hydrophobic aspirin is 0.1-10% of the combined level of EPA and DHA. Another embodiment is the combination of hydrophobic aspirin and hydrophobic sesamol in the 0.1-10% of the combined level of EPA and DHA. The EPA and DHA can be in the form of ethyl esters, triglycerides, free fatty acids, phospholipids, or other formats that are hydrophobic. The advantages of the hydrophobic derivatives of aspirin and sesamol is that they are compatible with the hydrophobic omega-3 fatty acids making it possible to have a highly defined composition suitable as a therapeutic drug for consistent clinical results.

In one embodiment, the combined amount of EPA and DHA ranges from 1 to 25 grams per day. In another embodiment, the amount of EPA and DHA is adjusted so that the arachidonic acid/eicosapentaenoic acid (AA/EPA) ratio in the blood is reduced to range between 1 and 10, and preferably between 1 and 5.

Reduction of chronic low-level inflammation may be achieved through concomitantly reducing the initiation phase and enhancing the resolution phase of the inflammation. In one embodiment, reducing the initiation phase is indicated by delivering adequate levels of omega-3 fatty acids combined with the appropriate level of hydrophobic aspirin and/or hydrophobic aspirin and hydrophobic sesamol to reduce the arachidonic acid/eicosapentaenoic acid (AA/EPA) ratio in the blood to range between 1 and 10, and preferably between 1 and 5. In another embodiment, enhancing the resolution phase of the invention is indicated by enhancing the level of pro-resolution mediators derived from both EPA and DHA in the blood by use of this invention.

The hydrophobic derivative may comprise a carboxylic derivative of aspirin. In one embodiment, the derivative of aspirin is obtained through esterification using an acyl chloride in the presence of pyridine.

In one embodiment, the acyl chloride is derived from a fatty acid. In another embodiment, the fatty acid comprises 2 to 22 carbon atoms and the degree of unsaturation of the fatty acid ranges from 0 to 6 double bonds.

In yet another embodiment, the acyl chloride is synthesized from a carboxyl derivative of methoxy polyethylene glycol characterized by CH₃O(CH₂CH₂O)ₙCH₂C(O)OH, wherein n ranges from 2 to 400.

In yet another embodiment, the carboxylic group is activated and the activated carboxylic group is an acid anhydride or obtained by reacting the carboxylic group with a 1,1 dicarbonyl diimidazole.

The hydrophobic derivative may comprise a carboxylic derivative of sesamol. In some embodiments, the sesamol may be derivatized with a fatty acid. The fatty acid may have a carbon chain comprising 2 to 22 carbon atoms.

In one embodiment, the hydrophobic derivative of sesamol is an acylated sesamol. In another embodiment, the hydrophobic sesamol is obtained by reacting a sesamol compound with an activated carboxylic acid. In yet another embodiment, the activated carboxylic acid is an acid chloride, an acid anhydride, or an acyl derivative obtained by reacting a carboxylic group moiety with 1,1 carbonyl diimidazole.

Additionally, the fatty acid may have a degree of unsaturation in the range of 0 to 6 double bonds per fatty acid molecule. In one embodiment, the activated carboxylic acid is an activated fatty acid. In another embodiment, the activated fatty acid comprises 2 to 22 carbon atoms and the degree of unsaturation of the activated fatty acid ranges from 0 to 6 double bonds.

In yet another embodiment, the activated fatty acid is an activated derivative of palmitic acid, oleic acid, linoleic acid, alpha-linolenic acid, arachidic acid, gadoleic acid, 5,8,11,14,17-eicosapentaenoic acid, or 4,7,10,13,16,19-docosahexaenoic acid.

In one embodiment, the derivative of sesamol is sesamol oleate. In another embodiment, the chemical derivative of the sesamol may comprise of a carboxylic acid derivative of polyethylene oxide. The polyethylene oxide may comprise 2 to 400 repeating units.

In some embodiments, the activated carboxylic acid is an acid chloride or an acid anhydride of CH₃O(CH₂CH₂O)ₙCH₂C(O)OH, or obtained by reacting CH₃O(CH₂CH₂O)ₙCH₂C(O)OH with 1,1 carbonyl diimidazole, wherein n ranges from 2 to 400.

The compositions of the invention may be prepared as liquid, a soft gelatin capsule, or an emulsion.

In one embodiment, the hydrophobic derivatives of aspirin and/or hydrophobic sesamol and hydrophobic aspirin are formulated in capsule, liquid or emulsion compatible with EPA and DHA formulations.

In some embodiments, the composition further comprises carotenoids such as lutein and zeaxanthin. In other embodiments, the composition further comprises additional fatty acids such as gamma-linolenic acid. In some embodiments, the composition can be further enhanced and combined with a balance of one or more proteins and one or more carbohydrates as a bar or meal replacement shake to help stabilize the levels of insulin that would otherwise increase the production of AA, wherein the proteins and the carbohydrates are present at a ratio of between about 0.5 and about 1.0.

The composition of the invention may be prepared for topical administration. For example, a topical composition can be a cream, gel, ointment, spray, lotion, solution, powder, or hard paste.

The compositions of the invention may be administered to human patients that require treatment for conditions associated with inflammation. Exemplary inflammation-associated conditions include, but are not limited to, obesity, metabolic syndrome, diabetes, cardiovascular disease, cancer, auto-immune disorders, ocular and neurological disorders. The compositions of the invention also may be administered to non-human mammals for veterinary purposes.

The hydrophobic derivative may include the embodiments described above. The composition may be administered via an enteral or parenteral route, or topically, and may comprise other biologically acceptable carriers, excipients, or diluents. Supplementary active ingredients also may be incorporated into the composition. The composition may be administered as a pharmaceutical, and may be prepared in various forms including, but not limited to, a liquid, a capsule, or an emulsion.

### BRIEF DESCRIPTION OF FIGURES

FIG. 1 shows the time course of the resolution of inflammation.

FIG. 2 illustrates the aspirin-mediated pathway for resolvins derived from EPA.

FIG. 3 illustrates the non-aspirin mediated pathways for resolvin formation from EPA and DHA.

FIG. 4 illustrates the metabolic pathways leading to the production of proinflammatory eicosanoids.

FIG. 5 shows the chemical structures of two embodiments (sesamol derivatives) of the invention.

FIG. 6 illustrates the synthesis and general chemical structure of the hydrophobic aspirin that can enhance the production of resolvins derived from omega-3 fatty acids.

### DETAILED DESCRIPTION OF THE INVENTION

The present invention is directed to compositions of hydrophobic derivatives of aspirin and/or sesamol that can be matched to the correct daily dosage of omega-3 fatty acids for the individual that allows for the maximum generation of pro-resolution mediators without the associated side effects such as increased bleeding when taken on a chronic basis to manage chronic low-level inflammation.

The clinical benefits of the present invention would allow precise administration of the compositions coupled with an easy-to-measure blood parameter to maintain adequate resolution of inflammation regardless of the cause. In addition, the dose of the administration can be individualized based on the type and severity of diseases, as well as gender and age of the subject, and can be used as either a therapeutic or prophylactic intervention.

The composition of the present invention consists of a defined amount of hydrophobic aspirin analog dissolved in given amount of a refined omega-3 concentrate containing high levels of both eicosapentaenoic acid (EPA) and docosahexaenoic acid (DHA). EPA and DHA are substrates required to make many of the most powerful pro-resolution mediators (resolvins, maresins, and protectins). From EPA comes E-series resolvins and from DHA comes D-series resolvins, maresins and protectins. The addition of a defined amount of hydrophobic aspirin will generate an increased production of pro-resolution mediators from these omega-3 fatty acids that constantly circulating in the blood. This invention overcomes the need for injection of synthetic resolvins into the blood to reach a target organ as each organ in the body will have a sufficient reservoir of omega-3 fatty acids in their membranes to maintain the constant generation of the necessary pro-resolution mediators.

The appropriate level of composition to be administered is determined by the AA to EPA ratio in the blood. AA is the primary driver of the eicosanoids that are important in the initiation process of inflammation. EPA is a stereochemical competitor to AA for access to a variety of enzymes that produce pro-inflammatory eicosanoids necessary for the initiation and continuation of the inflammatory response as well as a necessary substrate for certain pro-resolution mediators such as E-series resolvins. Thus the AA/EPA ratio in the blood allows one to optimize the level of the composition to be administered for either the therapeutic treatment or prophylactic maintenance to prevent excess inflammation with a precision simply not available by giving oral aspirin and omega-3 fatty acids separately, or by injections of synthesized pro-resolution mediators. The effect of the hydrophobic analog of aspirin can be further enhanced by the addition of a hydrophobic analog of sesamol that reduces the production of AA thus reducing the levels of omega-3 fatty acids needed for resolution.

It has been unexpectedly discovered that low levels of hydrophobic aspirin or the combination of hydrophobic derivatives of sesamol and hydrophobic aspirin with eicosapentaenoic acid (EPA) and docosahexaenoic acid (DHA) overcomes such an obstacle for the inconsistent therapeutic results using omega-3 fatty acids by achieving enhanced anti-inflammatory results at low levels of the added hydrophobic compounds.

### Increase in the Resolution of Chronic Low-Level Inflammation

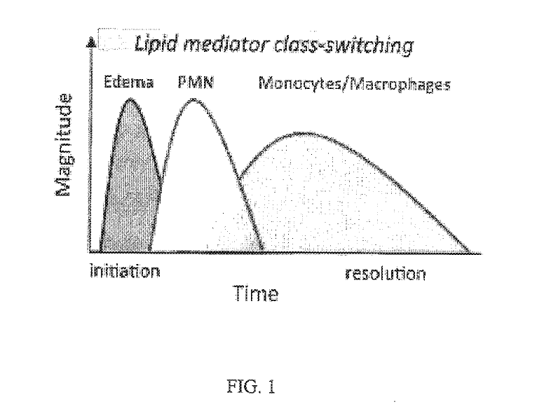

Chronic low-level inflammation is characterized in part by a failure to actively terminate the inflammation response when no longer required. The resolution phase begins with the decrease of the infiltration of polymorphonuclear leukocytes (PMN) into the site of inflammation and acceleration with the clearing of macrophages from the site of inflammation (FIG. 1). Resolvins are the primary mediators involved in this resolution process. Unless sufficient levels of resolvins are produced, the resolution phase is attenuated and chronic low-level inflammation remains at the site of inflammation.

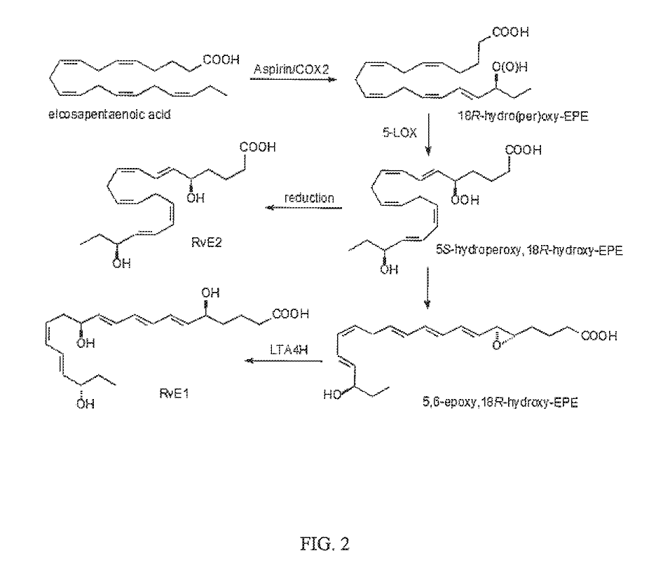

Low levels of aspirin are know to interact with COX enzymes to create modified COX enzymes that increase the production of pro-resolution mediators derived from EPA and DHA (FIG. 2).

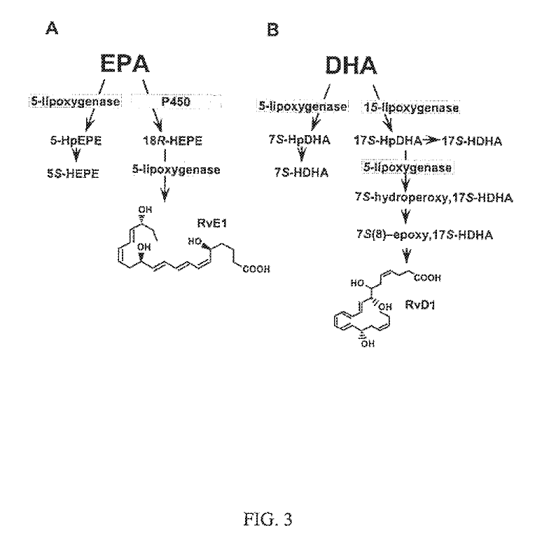

At high aspirin levels, the enhancement effect is saturated. Therefore there appears to be a discrete range of aspirin levels relative to the omega-3 fatty acids in which the generation of pro-resolution mediators from EPA and DHA is optimal. The use of hydrophobic aspirin combined in the same formulation with the administered omega-3 fatty acids allows one to optimize resolvin production thus enhancing the resolution phase of chronic low-level inflammation. This pathway is different from the less efficient pathway to generate resolvins that operates in the absence of aspirin (FIG. 3).

### Reduction of Chronic Low Level Inflammation

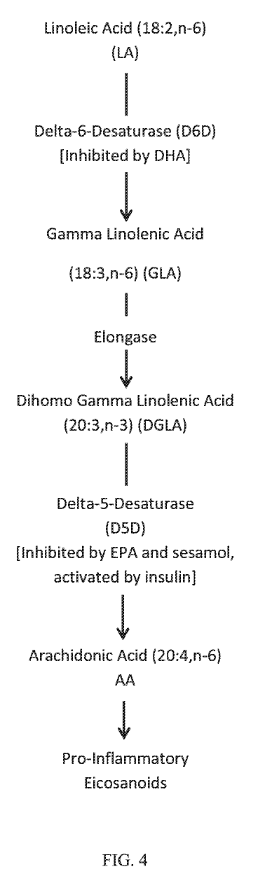

Chronic low-level inflammation is characterized by the constant maintenance of the initiation phase of inflammation, which starts with the increased production of pro-inflammatory eicosanoids derived from arachidonic acid (AA). This increase in inflammation is primarily mediated by increased expression of the COX-2 enzyme induced by the activation of the gene transcription factor NF-kB. As shown in FIG. 4, the enzyme Δ-5-desaturase (D5D) is required for converting dihomo gamma linolenic acid (DGLA) into arachidonic acid (AA). Thus, this constant generation of inflammation can be reduced by supplying an inhibitor of the enzyme Δ-5-desaturase (D5D) which otherwise converts dihomo gamma linolenic acid into AA. One such inhibitor is the small molecule sesamol. However, sesamol is not hydrophobic and therefore cannot be supplied simultaneously with the omega-3 fatty acids in either capsule, liquid or emulsion (both micro and macro) formulations. This can be overcome by the synthesis of hydrophobic derivatives of sesamol that are completely compatible with omega-3 fatty acid formulations. Such a hydrophobic sesamol would decrease the intensity of the initial pro-inflammatory phase by reducing the levels of AA.

However, like aspirin, sesamol is also not soluble in omega-3 fatty acid formulations. The invention addresses this deficiency by making hydrophobic derivatives of aspirin and/or combinations of hydrophobic derivatives of aspirin and sesamol that are compatible with omega-3 fatty acids in capsule, liquid, or emulsion (both micro and macro) formulations.

The development of such hydrophobic derivatives of sesamol and aspirin allows the development of precise ratios of the two compounds that can be precisely administered simultaneously with omega-3 fatty acids in a variety of delivery systems. For example, if the hydrophobic sesamol concentration is too high, this might reduce AA levels to an insufficient level to initiate a positive initiation response. This is also true for the hydrophobic aspirin as it has been shown that there is discrete range of aspirin that increases resolvin production and beyond that range, there is the potential of increased bleeding. Having hydrophobic derivatives of sesamol and aspirin combined with a precise level of administered omega-3 fatty acids reduces the possibility of not having enough aspirin to facilitate increased pro-resolution mediator formation, but not too much aspirin to increase potential bleeding which is also one of the potential side-effects of the use of high-dose omega-3 fatty acids.

### Sesamol Derivatives

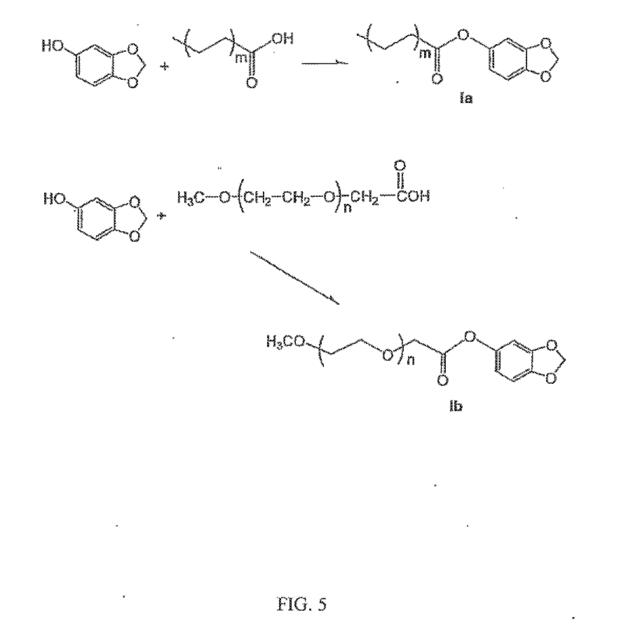

Sesamol is a chemical compound that has been shown to inhibit D5D activity. However, sesamol is incompatible with omega-3 fatty acid formulations as discussed above. The applicant has discovered that the free hydroxyl group of sesamol can be acylated with a suitable carboxylic acid moiety, including fatty acids and carboxylic derivatives of polyethylene oxide. FIG. 5 shows the chemical structures of two sesamol compounds derivatized with a generic fatty acid (compound Ia) and a carboxylic acid derivative of methoxy polyethylene oxide (compound Ib), respectively. The variables m and n may be an integer in the range of 1 to 11, and 2 to 400, respectively. Such derivatization helps to enhance the stability and bioavailability of the sesamol compound by attaching it to a biologically inert hydrophilic (i.e., polyethylene oxide) or hydrophobic (i.e., fatty acid) moiety.

Fatty acid derivatives of sesamol may be prepared from various fatty acids. Natural fatty acids, either isolated from natural sources or made synthetically, are preferred. Both saturated fatty acids and fatty acids with various degrees of unsaturation may be used, depending on the physical properties that one desires to impart to the invention. For instance, the fatty acid may have a degree of unsaturation in the range of 0 to 6 double bonds per fatty acid molecule. The fatty acid may be of various lengths and may have 2 to 22 carbon atoms per molecule. Fatty acid derivatives are hydrophobic in nature, and thus can be incorporated into circulating lipoproteins or cell membranes as a long-lived drug depot for sesamol. Examples of suitable fatty acids include, but are not limited to, palmitic acid, oleic acid, linoleic acid, alpha-linolenic acid, arachidic acid, gadoleic acid, 5,8,11,14,17-eicosapentaenoic acid, and 4,7,10,13,16,19-docosahexaenoic acid. Fatty acid derivatives of sesamol may be synthesized using standard organic chemistry via the activation of the carboxylic group with an acid chloride, an acid anhydride, or other activating agents such as 1,1 dicarbonyl diimidazole.

In alternative embodiments, a hydrophilic derivative of sesamol may be prepared by attaching a carboxylic acid derivative of polyethylene oxide to the sesamol molecule. As used herein, “a carboxylic acid derivative of methoxy polyethylene oxide” refers to a polymer of ethylene oxide with 2 to 400 repeating units having the terminal hydroxyl group converted into a carboxylic acid group. For instance, the other terminal group may be methoxylated. The process of attaching one or more chains of polyethylene oxide to a compound is often referred to as “pegylation.” In this case, the attachment of the hydrophilic polyethylene oxide moiety helps to increase the lifetime of the sesamol compound in the plasma compartment, which after the derivatization acts as a circulating depot. As the pegylated sesamol circulates in the plasma, sesamol is being slowly released into the system, which helps to reduce its toxicity.

### Aspirin Derivatives

Aspirin is a chemical compound that has been shown to increase resolvin activity. However, aspirin is incompatible with omega-3 fatty acid formulations as discussed above. Hydrophobic derivatives of aspirin as pro-drugs to reduce bleeding have already been described in the U.S. Pat. No. 8,486,974.

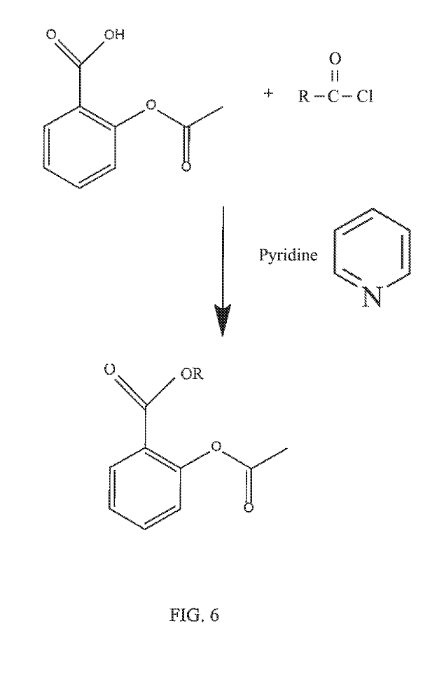

Hydrophobic derivatives of aspirin can be obtained through esterification by reacting an aspirin compound with an acyl chloride (FIG. 6). In one embodiment, the fatty acid is 2 to 22 carbon atoms. In an alternative embodiment, the acyl chloride is a carboxyl methoxy polyethylene glycol characterized by CH₃O(CH₂CH₂O)ₙCH₂C(O)OH, wherein n ranges from 2 to 400 that has been derivatized to an acyl chloride. Other embodiments may include making acid anhydrides or 1,1 carbonyl diimidazole derivatives of the carboxyl group.

### Formulation of the Composition

The level of the hydrophobic derivatives of each compound depends on the levels of omega-3 fatty acids in the final formulation. It is anticipated that the levels of the hydrophobic sesamol derivative will be approximately 0.1-10% of the total levels of EPA and DHA in the formulation. Likewise, it is anticipated that the levels of the hydrophobic aspirin will be in the levels of 0.1-10% of the levels of EPA and DHA in the formulation.

The anticipated dose of the invention would be a sufficient level of omega-3 fatty acids to reduce the AA/EPA ratio in the blood to range between 1 and 10, with the ideal range of between 1 and 5. The AA/EPA ratio in the Japanese populations is approximately 1.5, whereas in the American population the ratio is approximately 20. The levels of omega-3 fatty acids required to reach an AA/EPA ratio of less than 10 is generally in the range of between 1 and 5 grams of EPA and DHA on a daily basis with 2.5 to 25 grams of EPA and DHA per day being required to maintain an AA/EPA ratio between 1 and 5 to give more consistent therapeutic results. The benefits of this invention is that potentially lower levels of omega-3 fatty acids may be required to reach the desired AA/EPA ratio in the blood with improved synthesis of pro-resolution mediators. Since the half-life of administered omega-3 fatty acids in blood is approximately two days, single daily dosing should be sufficient to maintain a constant AA/EPA ratio. The dosing can be accomplished with soft gelatin capsule, liquid administration, or use of emulsion (both micro and macro) as well as other delivery systems known to one skilled in the art.

The benefits of the invention will be a more highly regulated ability of omega-3 fatty acids to control both the initiation and resolution phases of inflammation. As a result, it can be considered a significant advance in therapeutic reduction of chronic low-level inflammation that appears to be the underlying cause of a great number of chronic disease conditions.

### Delivery of the Composition

The composition may be delivered through traditional methods of administration such as via enteral, parenteral or topical routes. For enteral administration, the composition comprising the sesamol and aspirin hydrophobic derivatives may be formulated into a liquid, a soft gelatin capsule, emulsion or other methods known to those skilled in the art with or without other carriers, excipients, or diluents. Supplementary active ingredients also may be incorporated into the composition. In preferred embodiments, the composition comprising the sesamol or aspirin derivatives may be formulated into a soft gelatin capsule with an appropriate source of omega-3 fatty acids containing EPA and DHA (e.g., fish oil or omega-3 fatty acid concentrates).

Parenteral administration may be through intravenous or subcutaneous injections with a suitable emulsion, liposomal, or micellar structure for delivery. For a sesamol derivative comprising polyethylene oxide, the composition may be prepared as an aqueous solution, whereas if a fatty acid is used to derivatize the sesamol compound, the composition may be prepared as an emulsion, a liposome, or a micellar formation.

In some embodiments, the composition of the invention may be consumed with a food product designed to reduce and stabilize insulin thereby reducing a driving force for the conversion of linoleic acid into arachidonic acid. Preferably, the food product comprises between about 1 gram and about 60 grams of carbohydrate and between about 1 gram and about 40 grams of protein. More preferably, both protein and carbohydrate are present in the food product at a ratio of between about 0.5 and about 1.0 of protein to carbohydrate, inclusive. This ratio helps to lower secretion of insulin, thus reducing the activating impact that insulin has on D5D activity. Food products of the invention may be prepared in various forms including, but not limited to, food bars, pasta, breakfast cereals, confection products (e.g., ice creams and pastries), beverages (e.g., ready-to-drink mixes and shakes), convenience foods (e.g., a frozen meal), and shelf-stabilized meals. In yet another embodiment, the food products of the invention may be incorporated into a subject's dietary plan such as anti-inflammatory diet consisting of low-glycemic load carbohydrates, balanced with low-fat protein in the same carbohydrate to protein ratio and containing very limited amounts of omega-6 and saturated fatty acids.

In another embodiment, the composition of the invention may be administered topically. For example, a topical composition can be formulated as a cream, gel, ointment, spray, lotion, solution, powder, or hard paste.

The following examples are provided to illustrate further and to facilitate the understanding of the invention and are not intended to limit the invention.

### Example 1

### Synthesis of Acylated Sesamol Derivatives

Fatty acid is activated using a 1:1 molar amount of 1,1 carbonyl diimidazole in a dry benzene solution. The solution is taken to dryness at the completion of the activation. To the dried compound is added an equimolar amount of sesamol. The combined reactants are heated under vacuum at a low temperature for 1-2 hours. The completeness of the reaction is determined by thin layer chromatography. The acylated sesamol is then isolated by column chromatography to yield the isolated invention. The physical state of the invention depends on the chain length of the fatty acid and its degree of unsaturation.

### Example 2

### Synthesis of Sesamol Oleate

17.7 mmoles of oleic acid was dissolved in 40 ml of dry benzene. To the mixture was added 17.7 mmoles of 1,1 carbonyldiimidazole. The reaction to activate the oleic acid was continued at room temperature until vigorous evolution of carbon monoxide has ceased. The reaction was then driven to completion by driving off the excess benzene under vacuum at 60° C. To the neat activated oleic acid was added 21 mmoles of sesamol. The mixture was heated at 60° C. for 2 hours under vacuum with constant rotation. The crude reaction mixture was purified using 50 grams of silica gel 60 in 2×44 cm column eluting with hexane and increasing percentages of acetone. The fractions containing the active compound were collected and evaporated to dryness giving 10 mmoles of the sesamol oleate for a 56% yield. HPLC chromatography using a 98:2 cyclohexanone/isopropyl eluting solvent give a single component with greater than 90% purity.

### Example 3

### Synthesis of Polyethylene Oxide Sesamol Derivatives

Methoxy polyethylene oxide molecules of various chain lengths are oxidized by KMnO4 to yield a carboxylic acid derivative. The carboxylic acid derivative of methoxy polyethylene oxide is activated using a 1:1 molar amount of 1,1 carbonyl diimidazole in a dry benzene solution. The solution is taken to dryness at the completion of the activation. To the dried compound is added an equimolar amount of sesamol. The combined reactants are heated under vacuum at a low temperature for 1-2 hours. The completeness of the reaction is determined by thin layer chromatography. The acylated sesamol is then isolated by column chromatography to yield the isolated invention. The physical state of the invention depends on the chain length of the methoxy polyethylene oxide molecule.

### Example 4

### Reduction of the Level of Arachidonic Acid in a Mammal Using a Hydrophobic Sesamol Derivative

The ability of a sesamol derivative to reduce the level of arachidonic acid produced by Δ-5 desaturase (D5D) in a mammal was clearly demonstrated in an animal study conducted by me as herein summarized below.

To determine the ability of a sesamol derivative of the present invention to inhibit the formation of arachidonic acid, a two-week feeding study was done with two groups of mice with 8 mice in each group. We mixed 4.4 g of sesamol palmitate into 60 ml of safflower oil. This provided a concentration of sesamol palmitate of 73.3 mg/ml. The mice in the sesamol group received 500 μl each day (36.7 mg) via gavage of the sesamol palmitate compound in safflower oil whereas the control group received 500 μl of the safflower oil control. Both groups were maintained on a low-fat diet. 200 μl of blood was drawn at three time points (0, 1, and 2 weeks) for analysis of the fatty acid composition in the plasma.

Since the sesamol derivative, sesamol palmitate, inhibits the enzyme delta-5-desaturase that converts dihomo gamma linolenic acid (DGLA) to arachidonic acid (AA), we choose to look at the decrease in the AA/DGLA ratio as an indication of efficacy of sesamol palmitate in inhibiting the key enzyme. The percent difference between the AA/DGLA ratio in each rat was determined at the starting point and at days 7 and 14. The percent differences in the AA/DGLA per each rat in the active and placebo groups were then averaged. The results are shown in Table 2.

TABLE 2
Average % decrease in AA/DGLA Ratio in Plasma

| | Days 0-7 | Days 0-14 |
|---|---|---|
| Placebo | −15.4 | −12.2 |
| Sesamol Palmitate | −25.4 | −23.4 |

The average decrease in the AA/DGLA ratio in the active group was approximately double that of the placebo group indicating that inhibition of the delta-5-desaturase enzyme was taking place. Furthermore, most of the effects were observed in the first week.

### Example 5

### Synthesis of Palmitoyl Analog of Aspirin

To a solution of acetylsalicylic acid (40 g, 289.6 mmol) in anhydrous pyridine (170 ml) was added dropwise palmitoyl chloride (72.44 g, 0.91 eq.) at 0° C. under nitrogen with stirring. The reaction mixture was slowly warm up to room temperature overnight. Thin Layer Chromatography (TLC) analysis indicated the completion of the reaction. Pyridine was removed under vacuum. The residual crude product was dissolved in ethyl acetate, and the solution was washed with cold 2M HCl aqueous solution (2×500 ml) and deionized (DI) water (2×500 ml). The organic phase was dried with anhydrous sodium sulfate. After the filtration to remove the sodium sulfate, ethyl acetate was removed under vacuum to yield the crude aspirin palmitate. The crude product was crystallized twice in acetone-water to give the pure product as white crystal (65.0 g, 65.5% yield). Aspirin palmitate: ¹H NMR (CDCl₃): δ 0.88 (t, 3H), 1.26 (m, 24H), 1.75 (m, 2H), 2.62 (t, 2H), 7.13 (dd, 1H), 7.35 (m, 1H), 7.62 (m, 1H), 8.11 (dd, 1H) ppm. (MS-ESI); M+NH₄⁺, 394.5, M+Na⁺, 399.3.

### Example 6

### Synthesis of Oleyl Analog of Aspirin

To a solution of acetylsalicylic acid (17 g, 123.1 mmol) in anhydrous pyridine (35 ml) was added dropwise oleyl chloride (35.2 g, 0.95 eq.) at 0° C. under nitrogen with stirring. The reaction mixture was slowly warm up to room temperature overnight. TLC analysis indicated the completion of the reaction. Pyridine was removed under vacuum. The residual crude product was dissolved in ethyl acetate, and the solution was washed with cold 2M HCl aqueous solution (2×250 ml) and DI water (2×250 ml). The organic phase was dried with anhydrous sodium sulfate. After the filtration to remove the sodium sulfate, ethyl acetate was removed under vacuum to yield the crude aspirin oleate. The crude product was crystallized twice in acetone-water to give the pure product as white crystal (25.0 g, 53% yield). Aspirin oleate: ¹H NMR (CDCl₃): δ 0.88 (t, 3H), 1.26 (m, 20H), 1.75 (m, 2H), 2.00 (m, 4H), 2.64 (t, 2H), 5.34 (m, 2H), 7.13 (dd, 1H), 7.36 (m, 1H), 7.61 (m, 1H), 8.11 (dd, 1H) ppm. (MS-ESI); M+NH₄⁺, 420.6, M+Na⁺, 425.1.

### Example 7

### Synthesis of Lauroyl Analog of Aspirin

To a solution of acetylsalicylic acid (10 g, 72.4 mmol) in anhydrous pyridine (20 ml) was added dropwise lauroyl chloride (16.7 g, 1.0 eq.) at 0° C. under nitrogen with stirring. The reaction mixture was slowly warm up to room temperature overnight. TLC analysis indicated the completion of the reaction. Pyridine was removed under vacuum. The residual crude product was dissolved in ethyl acetate, and the solution was washed with cold 2M HCl aqueous solution (2×130 ml) and DI water (2×130 ml). The organic phase was dried with anhydrous sodium sulfate. After the filtration to remove the sodium sulfate, ethyl acetate was removed under vacuum to yield the crude aspirin laurate. The crude product was crystallized twice in acetone-water to give the pure product as white crystal (15.5 g, 67% yield). Aspirin laurate: ¹H NMR (CDCl₃): δ 0.88 (t, 3H), 1.26 (m, 16H), 1.75 (m, 2H), 2.64 (t, 2H), 7.13 (dd, 1H), 7.36 (m, 1H), 7.63 (m, 1H), 8.12 (dd, 1H) ppm. (MS-ESI); M+NH₄⁺, 338.2, M+Na⁺, 343.4.

### Example 8

### Synthesis of Palmitoyl Analog of Sesamol

To a solution of sesamol (13.5 g, 97.7 mmol) in anhydrous pyridine (20 ml) was added dropwise palmitoyl chloride (25.2 g, 0.95 eq.) at 0° C. under nitrogen with stirring. The reaction mixture was slowly warm up to room temperature overnight. TLC analysis indicated the completion of the reaction. Pyridine was removed under vacuum. The residual crude product was dissolved in ethyl acetate, and the solution was washed with cold 2M HCl aqueous solution (2×130 ml) and DI water (2×130 ml). The organic phase was dried with anhydrous sodium sulfate. After the filtration to remove the sodium sulfate, ethyl acetate was removed under vacuum to yield the crude sesamol palmitate. The crude product was crystallized twice in acetone-water to give the pure product as white crystal (25.6 g, 73% yield). Sesamol Palmitate: ¹H NMR (CDCl₃): δ 0.88 (t, 3H), 1.26 (m, 24H), 1.73 (m, 2H), 2.52 (t, 2H), 5.98 (S, 2H), 6.52 (m, 1H), 6.59 (m, 1H), 6.77 (m, 1H) ppm. (MS-ESI); M+NH₄⁺, 394.4.

### Example 9

### Increased Generation of a Resolvin in a Mammal by a Hydrophobic Aspirin Derivative

Three different oil mixtures were prepared, each with 10 ml of oil. The first material used to test the invention consisted of 10 ml of refined omega-3 concentrates containing 4 grams of EPA and 2 grams of DHA and 20 mg of the oleyl hydrophobic analog of aspirin. The second consisted of 10 ml of refined omega-3 concentrates containing 4 grams of EPA and 2 grams of DHA and no hydrophobic aspirin. The third consisted of 10 ml of corn oil containing 5.5 grams of linoleic acid and no EPA and DHA. Each of the 10 ml samples of oil mixture were then diluted to 400 ml with refined olive oil. All mixing was done under nitrogen.

Mice were divided into three groups and each group was given 160 ul of the diluted oils by gavage on daily basis for two weeks. The first group (Group 1) of five mice received the omega-3 concentrate and oleyl analog of aspirin. The second group (Group 2) of five mice received the same levels of EPA and DHA as Group 1, but without the oleyl derivative of aspirin. The third group (Group 3) of six mice received the corn oil. All groups were fed with a standard diet for two weeks.

At the end of two weeks, blood samples were taken from the animals and the plasma containing serum lipoproteins was separated from the red blood cells and other cells by centrifugation. The isolated plasma was then assayed for the levels of resolvin D1 (RvD1) using an ELISA assay from Cayman Chemicals. The plasma was also analyzed by GC-FID for the fatty acid levels as percent of the total integrated area of all the fatty acids. The ratio of AA/EPA in the plasma in each sample was calculated from the individual areas under the curve for AA and EPA when analyzed using a gas chromatograph with a flame ionization detector. The results are shown in Table 3.

TABLE 3
Average Increase in Levels of RvD1 in Plasma

| | RvD1 levels | AA/EPA ratio |
|---|---|---|
| Group | (pg/ml) | (plasma) |
| Fish oil + hydrophobic aspirin | 795 | 4.8 |
| Fish oil alone | 674 | 4.4 |
| Corn oil alone | 520 | 6.5 |

The data indicates that the omega-3 fatty acids were being incorporated into the serum as indicated by lower AA/EPA compared to the corn oil controls. Although the fish oil only group had a lower AA/EPA ratio compared to the fish oil plus hydrophobic aspirin group, the levels of RvD1 were 16% higher indicating greater conversion of DHA into RvD1 and 52% higher than the corn oil control.

This is further illustrated in Table 4 which shows measured AA/EPA ratio in the isolated plasma phospholipids (PL) in the serum, which is a better indicator of the fatty acid composition in the serum lipoproteins.

TABLE 4
Average Levels of RvD1 in Plasma Relative to the AA/EPA ratio

| | RvD1 levels | AA/EPA ratio |
|---|---|---|
| Group | (pg/ml) | (isolated PL) |
| Fish oil + hydrophobic aspirin | 795 | 5.0 |
| Fish oil alone | 674 | 4.3 |
| Corn oil alone | 520 | 7.3 |

Even through the AA/EPA ratio was lower in the omega-3 fatty acid only group compared to the group with the omega-3 fatty acids and hydrophobic aspirin, the generation of RvD1 in the plasma was higher in the group without the hydrophobic aspirin.

### Example 10

A one-gram soft gelatin capsule containing 0.4 grams of EPA and 0.2 grams of DHA, and 3 mg of hydrophobic aspirin is prepared. 15 such soft gelatin capsules will be given orally to reduce the AA/EPA ratio in the plasma to 1-5 necessary for the optimal reduction of chronic low-grade inflammation associated with chronic conditions including obesity, metabolic syndrome, diabetes, cardiovascular disease, cancer, auto-immune disorders, ocular and neurological disorders.

### Example 11

An emulsion containing 8 grams of EPA and 4 grams of DHA, 30 mg of hydrophobic aspirin derivative and 30 mg of hydrophobic sesamol derivative is prepared. This emulsion will be given orally to reduce the AA/EPA ratio for the reduction of chronic low-grade inflammation associated with chronic conditions including obesity, metabolic syndrome, diabetes, cardiovascular disease, cancer, auto-immune disorders, ocular and neurological disorders.

### Example 12

The efficacy of the composition can be increased by lowering and stabilizing insulin levels as a meal replacement. A solid food format in a bar form containing 14 grams of protein, 21 grams of low-glycemic carbohydrates, and 7 grams of fat will be co-administered with the composition. The bar can be fortified with vitamins and minerals.

### Example 13

The efficacy of the composition can be increased by lowering and stabilizing insulin levels using a shake as meal replacement. A shake containing 25 grams of protein, 39 grams of low-glycemic carbohydrates, and 13 grams of fat consisting of primarily monounsaturated fats will be co-administered with the composition as a meal replacement. The shake can be fortified with vitamins and minerals.

Variations, modifications, and other implementations of what is described herein will be occur to those of ordinary skill in the art without departing from the spirit and the essential characteristics of the invention. Accordingly, the scope of the invention is to be defined not by the preceding illustrative description but instead by the following claims, and all changes that come within the meaning and range of equivalency of the claims are intended to be embraced therein.

What is claimed is:

1. A composition comprising hydrophobic derivatives of aspirin and/or hydrophobic derivatives of sesamol in combination with eicosapentaenoic acid (EPA) and docosahexaenoic acid (DHA), for reducing chronic low-level inflammation associated with chronic conditions including obesity, metabolic syndrome, diabetes, cardiovascular disease, cancer, auto-immune disorders, ocular and neurological disorders in a mammal.

2. The composition of claim 1, wherein the EPA and DHA can be in the form of ethyl esters, triglycerides, free fatty acids, phospholipids, or other formats that are hydrophobic.

3. The composition of claim 1, wherein the level of hydrophobic aspirin is 0.1-10% of the combined level of EPA and DHA.

4. The composition of claim 1, wherein combined the level of hydrophobic aspirin and hydrophobic sesamol is 0.1-10% of the combined level of EPA and DHA.

5. The composition of claim 1, wherein reduction of inflammation is through concomitantly reducing the initiation phase and enhancing the resolution phase of the inflammation.

6. The composition of claim 1, wherein the amount of EPA and DHA is adjusted so that the arachidonic acid/eicosapentaenoic acid (AA/EPA) ratio in the blood is reduced to range between 1 and 10, and preferably between 1 and 5.

7. The composition of claim 5, wherein enhancing the resolution phase is indicated by enhancing the level of resolvins in the blood.

8. The composition of claim 1, wherein the amount of EPA and DHA ranges from 1 to 25 grams per day.

9. The composition of claim 1, wherein the derivative of sesamol is an acylated sesamol.

10. The composition of claim 9, wherein the acylated sesamol is obtained by reacting a sesamol compound with an activated carboxylic acid.

11. The composition of claim 10, wherein the activated carboxylic acid is an acid chloride, an acid anhydride, or obtained by reacting a carboxylic acid with 1,1 carbonyl diimidazole.

12. The composition of claim 10, wherein the activated carboxylic acid is an activated fatty acid.

13. The composition of claim 12, wherein the activated fatty acid comprises 2 to 22 carbon atoms.

14. The composition of claim 10, wherein the degree of unsaturation of the activated fatty acid ranges from 0 to 6 double bonds.

15. The composition of claim 10, wherein the activated fatty acid is an activated derivative of palmitic acid, oleic acid, linoleic acid, alpha-linolenic acid, arachidic acid, gadoleic acid, 5,8,11,14,17-eicosapentaenoic acid, or 4,7,10,13,16,19-docosahexaenoic acid.

16. The composition of claim 1, wherein the derivative of sesamol is sesamol oleate.

17. The composition of claim 10, wherein the activated carboxylic acid is an acid chloride or an acid anhydride of CH₃O(CH₂CH₂O)ₙCH₂C(O)OH, or obtained by reacting CH₃O(CH₂CH₂O)ₙCH₂C(O)OH with 1,1 carbonyl diimidazole, wherein n ranges from 2 to 400.

18. The composition of claim 1, wherein the derivative of aspirin is obtained through esterification with an acyl chloride.

19. The composition of claim 18, wherein the esterification is carried out by reacting an aspirin compound with an acyl chloride.

20. The composition of claim 19, wherein the acyl chloride is derived from a fatty acid.

21. The composition of claim 20, wherein the fatty acid comprises 2 to 22 carbon atoms.

22. The composition of claim 21, wherein the degree of unsaturation of the fatty acid ranges from 0 to 6 double bonds.

23. The composition of claim 19, wherein the acyl moiety is a carboxylic derivative of methoxy polyethylene glycol.

24. The composition of claim 23, wherein the carboxyl methoxy polyethylene glycol compound is characterized by CH₃O(CH₂CH₂O)ₙCH₂C(O)OH, wherein n ranges from 2 to 400.

25. The composition of claim 1, wherein the composition is prepared as a pharmaceutical, a liquid, a soft gelatin capsule, or an emulsion.

26. The composition of claim 1, wherein the hydrophobic derivatives of sesamol and aspirin are formulated in capsule, liquid or emulsion compatible with EPA and DHA formulations.

27. The composition of claim 1, wherein the composition further comprises carotenoids such as lutein and zeaxanthin.

28. The composition of claim 1, wherein the composition further comprises gamma-linolenic acid ester.

29. The composition of claim 1, wherein the composition is co-administered with a food product containing proteins and carbohydrates, wherein the proteins and the carbohydrates are present at a ratio of between about 0.5 and about 1.0.

30. The composition of claim 1, wherein the composition is prepared for topical administration.

31. A method for reducing chronic low-level inflammation associated with chronic conditions including obesity, metabolic syndrome, diabetes, cardiovascular disease, cancer, auto-immune disorders, ocular and neurological disorders in a mammal, comprising administering to a mammal a composition comprising hydrophobic derivatives of aspirin and/or hydrophobic derivatives of aspirin and sesamol in combination with eicosapentaenoic acid (EPA) and docosahexaenoic acid (DHA).

32. The method of claim 31, wherein the level of hydrophobic aspirin is 0.1-10% of the combined level of EPA and DHA.

33. The method of claim 31, wherein the combined level of hydrophobic aspirin and sesamol is 0.1-10% of the combined level of EPA and DHA.

34. The method of claim 31, wherein reduction of inflammation is through concomitantly reducing the initiation phase and enhancing the resolution phase of the inflammation.

35. The method of claim 31, wherein the amount of EPA and DHA is adjusted so that the arachidonic acid/eicosapentaenoic acid (AA/EPA) ratio in the blood is reduced to range between 1 and 10, and preferably between 1 and 5.

36. The method of claim 34, wherein enhancing the resolution phase is indicated by enhancing the level of resolvins in the blood.

37. The method of claim 31, wherein the amount of EPA and DHA ranges from 1 to 25 grams per day.

38. The method of claim 31, wherein the derivative of sesamol is an acylated sesamol.

39. The method of claim 38, wherein the acylated sesamol is obtained by reacting a sesamol compound with an activated carboxylic acid.

40. The method of claim 39, wherein the activated carboxylic acid is an acid chloride, an acid anhydride, or obtained by reacting a carboxylic acid with 1,1 carbonyl diimidazole.

41. The method of claim 39, wherein the activated carboxylic acid is an activated fatty acid.

42. The method of claim 41, wherein the activated fatty acid comprises 2 to 22 carbon atoms.

43. The method of claim 41, wherein the degree of unsaturation of the activated fatty acid ranges from 0 to 6 double bonds.

44. The method of claim 39, wherein the activated fatty acid is an activated derivative of palmitic acid, oleic acid, linoleic acid, alpha-linolenic acid, arachidic acid, gadoleic acid, 5,8,11,14,17-eicosapentaenoic acid, or 4,7,10,13,16,19-docosahexaenoic acid.

45. The method of claim 31, wherein the derivative of sesamol is sesamol oleate.

46. The method of claim 39, wherein the activated carboxylic acid is an acid chloride or an acid anhydride of CH₃O(CH₂CH₂O)ₙCH₂C(O)OH, or obtained by reacting CH₃O(CH₂CH₂O)ₙCH₂C(O)OH with 1,1 carbonyl diimidazole, wherein n ranges from 2 to 400.

47. The method of claim 31, wherein the derivative of aspirin is obtained through esterification with an acyl chloride

48. The method of claim 47, wherein esterification is carried out by reacting an aspirin compound with an acyl chloride.

49. The method of claim 48, wherein the acyl chloride is derived from a fatty acid.

50. The method of claim 49, wherein the fatty acid comprises 2 to 22 carbon atoms.

51. The method of claim 50, wherein the degree of unsaturation of the fatty alcohol ranges from 0 to 6 double bonds.

52. The method of claim 48, wherein the acyl group is a carboxyl derivative of methoxy polyethylene glycol.

53. The method of claim 52, wherein the carboxyl derivative of methoxy polyethylene glycol is characterized by CH₃O(CH₂CH₂O)ₙCH₂C(O)OH, wherein n ranges from 2 to 400.

54. The method of claim 31, wherein the composition is prepared as a pharmaceutical in a form selected from the group consisting of a liquid, a capsule, or an emulsion.

55. The method of claim 31, wherein the hydrophobic derivatives of sesamol and aspirin are formulated in capsule, liquid or emulsion compatible with EPA and DHA formulations.

56. The method of claim 31, wherein the composition further comprises carotenoids such as lutein and zeaxanthin.

57. The method of claim 31, wherein the composition further comprises gamma-linolenic acid ester.

58. The method of claim 31, wherein the composition is co-administered with a food product, wherein the proteins and the carbohydrates are present at a ratio of between about 0.5 and about 1.0.

59. The method of claim 31, wherein the administering step is carried out enterally or parenterally.

60. The method of claim 31, wherein the composition is administered to the mammal in an amount that is effective to reduce inflammation in the mammal.

61. The method of claim 31, wherein the composition can be administered in soft gelatin capsule, liquid administration or emulsion (both micro and macro), as well as in other delivery systems.

62. The method of claim 31, wherein the composition can be administered in a single daily dose.

63. The method of claim 62 wherein the single daily dose comprises 20 mg of hydrophobic aspirin.

64. The method of claim 31, wherein the composition is prepared for topical administration.
